(19) United States
(12) Patent Application Publication
(10) Pub. No.: US 2026/0283777 A1
(43) Pub. Date: Sep. 24, 2026

(54) Ureteral Stent And Methods Therefor

(71) Applicant: JMT Medical, Inc., Billings, MT (US)

(72) Inventor: Jeffrey Justin Rentz, Billings, MT (US)

(73) Assignee: JMT Medical, Inc., Billings, MT (US)

(21) Appl. No.: 19/573,210

(22) Filed: Mar. 20, 2026

Related U.S. Application Data
(60) Provisional application No. 63/775,910, filed on Mar. 21, 2025.

(51) Int. Cl. A61F 2/04; A61F 2/00; A61F 2/95

(52) U.S. Cl. CPC A61F 2/04; A61F 2002/0081; A61F 2002/048; A61F 2002/9505; A61F 2210/0057; A61F 2220/0008; A61F 2230/0091; A61F 2250/0067

(57) ABSTRACT

A ureteral stent is made from a polymeric tubular body that is configured to allow for retention in the ureter of a subject by helical engagement with and radial extension of the ureter over substantially the entire length of the stent. In such stents, the polymeric material has a Young's modulus/spring constant that is matched to the Young's modulus of the ureter such that, upon placement of the stent, the ureter is radially expanded in an amount of between 2% and 20% (as measured form the uncontracted state) while still allowing peristaltic movement along the ureter.

This application claims priority to our copending U.S. Provisional Patent Application with the Ser. No. 63/775,910, filed Mar. 21, 2025, which is incorporated by reference herein in its entirety.

### FIELD OF THE INVENTION

The field of the invention is devices, kits, and methods related to surgery, and especially as it relates to devices and methods of placing a stent in the ureter between the kidney and the bladder.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

There are various ureteral stents known in the art, with many of them having a straight central portion between two terminal retention elements. One retention element in such stents is disposed in the bladder while the other retention element is disposed in the renal pelvis, and each of the retention elements may have a distinct geometry. For example, WO 2018/156650 discloses a stent having two ‘pigtail’ retention elements, and U.S. Pat. No. 4,531,933 teaches two perpendicular coil retention elements. In other examples, a single perpendicular coil retention element is shown in US 2003/0181842, and U.S. 7,037,345 discloses a coil retention element on one end and a ‘pigtail’ retention element on the other end. In still further examples, a ureteral stent has a serpentine retention element on one end and a ‘pigtail’ retention element as described in U.S. Pat. No. 4,643,716. Where desired, a bladder retention element may be relatively small and configured as a loop as described in EP 1792636. While such stents may provide at least some functionality, various problems nevertheless remain. Most significantly, as ureteral peristalsis is an involuntary and continuous contraction along the length of the ureter, the central portion of the stent in the ureter will impact stones against the ureteral wall resulting in spasms and significant pain. Moreover, the retention elements may cause discomfort, particularly in the pressure sensitive portions of the bladder and/or where patient movement causes shifting of the retention elements.

In still further known examples of stents, US 2003/0040803 discloses a coiled stent with polymeric cover for the prostatic urethra. However, such stent is unsuitable for use in the ureter between the kidney and the bladder. US 2008/0262599 teaches a stent with spiral geometry that is made from an expanding metallic mesh. While conceptually interesting, such stent entirely fails to accommodate for peristaltic movement in the ureter. Similarly, as shown in U.S. Pat. No. 5,554,189, a closely coiled metallic stent is described, allowing for bending deformation and limited lateral extension. Once more, however, flexibility to accommodate peristaltic motion of the ureter is not provided for in such stents. In still other examples, a dual layer coiled stent structure is described in U.S. Pat. No. 10,517,710. While allowing flexibility for bending motion, migration of such stent structure nevertheless remains a problem and so requires retention elements such as a pigtail structure on both termini. Similarly, U.S. Pat. No. 11,213,414 teaches stent structures with controlled longitudinal extension. Once more, such stent structure will require terminal retention structures.

In yet another example, U.S. Pat. No. 7,044,981 teaches a ureteral stent that does not extend into the renal pelvis but is retained in the ureter by two self-anchoring ends, a regular helical coil at the proximal end near the kidney and a spiral ‘pigtail’ at the distal end in the bladder. Located between the retention elements is an irregular helical portion having a distal region with a non-circular cross section to reduce irritation and discomfort, which terminates in the spiral ‘pigtail’ at the distal end. However, due to the complex configuration of the stent, manufacture is difficult. More significantly, the stent configuration in the '981 patent permits ‘self-adjustment during patient movement’ allowing movement and even shifting of the proximal end of the stent along the ureter. However, and beyond frictional irritation, such permitted movement may be particularly problematic as peristaltic contractions along the ureter will tend to force the stent in a distal direction, thereby repeatedly impacting the helical coil at the proximal end of the ureter, potentially leading to significant discomfort or pain. Likewise, U.S. Pat. No. 4,813,925 teaches a stent configuration that has a spiral portion with terminal retention elements in which the spiral portion forces the ureter into an opened shape, thus restoring patency of a previously collapsed ureter. Once more, such spiral structure is generally not able to flex with ureteral peristaltic motion and is likely to induce significant patient discomfort and even spasms.

Thus, even though various devices and methods of stent placement in the ureter are known in the art, all or almost all of them suffer from several drawbacks, particularly where stones at least partially obstruct the flow of urine and/or where peristaltic motion moves the stent within the ureter. Therefore, there remains a need for improved devices and methods of ureteral stents.

### SUMMARY OF THE INVENTION

The inventive subject matter is directed to various devices, methods, and kits for improved ureteral stents that do not require retention elements at either end and that are placed and retained in the ureter in a manner that permits and conforms to peristaltic motion while at the same time avoiding impaction of stones against the luminal wall of the ureter. Moreover, the ureteral stents presented herein will, upon deployment into the ureter, retain patency of the ureter and form a composite fluid path comprising an intrahelical fluid path and an intratubular fluid path.

In one aspect of the inventive subject matter, the inventor contemplates a ureteral stent that includes a tubular body having a proximal portion with a proximal end and a distal portion with a distal end. Most typically, the tubular body is deformable between a tensioned linear delivery configuration and an expanded helical deployment configuration, wherein the tubular body comprises a polymeric material having a Young's modulus that is effective to (a) exert a frictional force on a delivery device in the tensioned linear delivery configuration to so retain the tubular body on the delivery device; and (b) exert a radially expansive force on an inner wall of a ureter in the expanded helical deployment configuration to radially expand the inner wall of the ureter to so retain the tubular body in the ureter. It is further contemplated that the stents presented herein will preferably have a plurality of fenestrations in the tubular body.

Where desired, wherein the proximal and/or distal portion may further comprise a non-helical portion. For example, the non-helical portion may comprise a retention element that is configured for placement into the pelvis of a kidney or into the bladder. Among other options, the non-helical portion may comprise, upon placement in the pelvis of the kidney, a coil configuration may be perpendicularly oriented to the longitudinal axis of the helical portion.

In further embodiments, the stent exerts the radially expansive force over a cumulative length of at least 75%, or at least 90%, of the overall length of the stent. Additionally, it is contemplated that the stent may be configured to radially expand the inner wall of the ureter by no more than 20% (e.g., radially expansion of the inner wall of the ureter by at least 2% and by no more than 8%). Therefore, in further exemplary embodiments it is contemplated that the ratio of the Young's modulus of the polymeric material to the Young's modulus of the ureter for radial expansion is between 1.2:1 and 1.9:1. Viewed from a different perspective, it is contemplated that the ratio of the Young's modulus of the polymeric material to the Young's modulus of the ureter for radial expansion may be selected such that, upon deployment of the stent, the inner wall diameter of the ureter is increased by between 3% and 10%.

Thus, in at least some embodiments it is contemplated that the Young's modulus of the polymeric material is between 2.5 MPa and 4.5 MPa, and/or that the ratio of the spring constant for radial expansion of the ureteral stent and the spring constant for radial expansion of the ureter is between 3:1 and 10:1. For example, it is contemplated that the spring constant for radial expansion of the ureter is between 2,000 N/m and 25,000 N/m, and/or that the spring constant for radial expansion of the ureteral stent is between 20,000 N/m and 60,000 N/m.

In still further embodiments it is contemplated that the stent does not comprise a retention element extending away from the tubular body. Where desirable, the stent may also include a radiopaque marker on the proximal and/or distal end. In some exemplary embodiments, the stent has an outer diameter between 2 mm (6 French) and 3.33 mm (10 French) in the tensioned linear delivery configuration. For example, the stent may have an outer diameter of 2.67 mm (8 French) in the tensioned linear delivery configuration. As will be readily appreciated, contemplated ureteral stents may have an overall length of between 150 mm and 300 mm in the linear delivery configuration (e.g., at least 160 mm in the linear delivery configuration such as 175 mm, or 245 mm, or 280 mm) to so accommodate use in children and adults of different height.

Additionally, it is contemplated that the stents presented herein may, in the helical deployment configuration, form a helix with an inner open diameter of between 1 mm and 5 mm and/or an outer helical diameter between 5 mm and 16 mm. Most typically, and depending on the overall length, typical contemplated stents may have between 4 and 8 complete helical turns. For example, contemplated stents may have between 4 and 6 complete helical turns and a length of between 150-200 mm, or have between 5 and 7 complete helical turns and a length of between 220-270 mm, or have between 6 and 8 complete helical turns and a length of between 250-300 mm. Moreover, it should be appreciated that the stent, in the helical deployment configuration, will form a composite fluid path comprising an intrahelical fluid path and an intratubular fluid path.

In still further embodiments, the polymeric material in contemplated stents may comprise a low-density polyethylene. Moreover, the polymeric material further comprises a pharmaceutical agent (e.g., an antimicrobial agent, a chemotherapeutic agent, an anti-inflammatory agent, an immune modulatory agent, and/or a radioactive agent).

Therefore, and viewed from yet another different perspective, the inventor also contemplates a ureteral stent that comprises a polymeric tubular body having a helical portion that is configured to helically engage with and radially extend a ureter into which the stent is placed. In such stents, the polymeric material of the helical portion has a Young's modulus that is matched to a Young's modulus for radial expansion of the ureter such that radial expansion of the ureter by the helical portion is in an amount of between 2% and 20%, and the Young's modulus of the polymeric material of the helical portion allows for elastic deformation of the helical portion into a linear delivery configuration.

For example, the ratio of the Young's modulus of the polymeric material of the helical portion to the Young's modulus of the ureter for radial expansion may be between 1.2:1 and 1.9:1. Thus, the Young's modulus of the polymeric material of the helical portion may be between 2.5 MPa and 4.5 MPa. Where desired, the polymeric tubular body may further include a plurality of fenestrations. As noted earlier, it is also contemplated that the stent may form, upon insertion into the ureter, a composite fluid path comprising an intrahelical fluid path and an intratubular fluid path.

In further contemplated aspects of such stents, the helical portion, without placement into the ureter, may have a helical outer diameter that is larger than an inner diameter of the ureter. Moreover, it is contemplated that such stents may have between 4 and 8 complete helical turns. Where desired, contemplated stents will also comprise a radiopaque marker at one or both terminal portions. Additionally, it is contemplated that the stent may further comprise a non-helical portion and/or a retention element that is configured for placement into a pelvis of a kidney or a bladder. For example, the non-helical portion comprises, upon placement in the pelvis of the kidney, a coil configuration that is perpendicularly oriented to a longitudinal axis of the helical portion. Most typically, the helical portion will have a length of between 150 mm and 300 mm, and/or the polymeric tubular body has an inner diameter between 0.2 mm and 4.0 mm.

Viewed from yet another different perspective, the inventor therefore also contemplates a ureteral stent ureteral stent that includes a polymeric tubular body having a helical portion that is configured to helically engage with and radially extend a ureter into which the stent is placed. In such stent, the helical portion has a spring constant that is matched to a spring constant for radial expansion of the ureter such that radial expansion of the ureter by the helical portion is in an amount of between 2% and 20%, and the spring constant of the helical portion allows for elastic deformation of the helical portion into a linear delivery configuration.

In some embodiments, the ratio of the spring constant for radial expansion of the helical portion and the spring constant for radial expansion of the ureter is between 3:1 and 10:1. Thus, suitable spring constants of the helical portion may be between 20,000 N/m and 60,000 N/m, and/or suitable spring constants for radial expansion of the ureter may be between 2,000 N/m and 25,000 N/m.

Where desired, the polymeric tubular body further comprises a plurality of fenestrations, and optionally further comprises a non-helical portion. As will be readily appreciated, the stent will form, upon insertion into the ureter, a composite fluid path comprising an intrahelical fluid path and an intratubular fluid path. Moreover, it is contemplated that the helical portion, before placement into the ureter, has a helical outer diameter that is larger than an inner diameter of the ureter. As noted above, contemplated stents may have between 4 and 8 complete helical turns.

Consequently, the inventor contemplates a method of placing a ureteral stent that includes a step of providing a delivery device to which a ureteral stent is concentrically coupled via frictional force in a tensioned linear delivery configuration. Most typically, the ureteral stent has a tubular body having a proximal end and a distal end, and the tubular body is deformable between the tensioned linear delivery configuration and an expanded helical deployment configuration. In a further step, the delivery device and stent in the tensioned linear delivery configuration are advanced into the ureter to a delivery location, and upon reaching the delivery location, the delivery device is withdrawn while the ureteral stent is maintained at the delivery location. At this point, the ureteral stent converts from the tensioned linear delivery configuration into the expanded helical deployment configuration, wherein the ureteral stent in the expanded helical deployment configuration exerts a radially expansive force on an inner wall of a ureter that radially expands the inner wall of the ureter to so retain the tubular body in the ureter while allowing for peristalsis of the ureter.

In some embodiments, the step of advancing may comprise retrograde advancing of the delivery device and stent through the urethra and bladder (typically using a guidewire). In further embodiments, the step of advancing may also comprise percutaneous antegrade advancing of the delivery device and stent through the kidney. As will be readily appreciated, and where appropriate, the delivery device may be advanced over a guide wire. Most typically, the ureteral stent in contemplated methods will be a ureteral stent as presented herein.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary schematic illustration of the guide wire inserted through the bladder into the kidney by the cystoscope.

FIG. 2 is an exemplary schematic illustration of the canula and stent over the guide wire that has been inserted through the bladder into the kidney.

FIG. 3 is an exemplary schematic illustration of the spiral conformal stent inserted into the kidney, ureter and bladder. The mechanical biology of the stent is in fact a spring that expands the ureter tube by a percentage that holds the stent in place and is conformal to the biology of the ureter tube. The ureter tube has a circumference Young's modulus of 2.31 MPa +/−0.46, whereas the stent spring has a percentage MPa greater than the ureter tube thereby holding the stent in place with a slightly larger outside spiral diameter when compared to the ureter tube. The Hook's Spring law constant of the ureter tube in that the stent is essentially a gentle spring holding the stent in place. There is a string at the end of the stent for easy removal.

FIG. 4 is an exemplary schematic illustration of the spiral conformal stent inserted into the kidney, ureter and bladder. This is a version that has a larger “J” spiral in the kidney to more firmly hold the stent in place The mechanical biology of the stent is in fact a spring that expands the ureter tube by a percentage that holds the stent in place and is conformal to the biology of the ureter tube. The ureter tube has a circumference Young's modulus of 2.31 MPa +/−0.46, whereas the stent spring has a percentage MPa greater than the ureter tube thereby holding the stent in place with a slightly larger outside spiral diameter when compared to the ureter tube. The Hook's Spring law constant of the ureter tube in that the stent is essentially a gentle spring holding the stent in place. There is a string at the end of the stent for easy removal.

FIG. 5 is an exemplary schematic illustration of the canula removed and the stent is now in place.

FIG. 6 is an exemplary schematic illustration of the complete 175 mm ureteral stent kit.

FIG. 7 is an exemplary schematic illustration of the complete 245 mm ureteral stent kit.

FIG. 8 is an exemplary schematic illustration of the complete 280 mm ureteral stent kit.

FIG. 9 is an exemplary schematic illustration of the “J” spiral stent showing the three diameters of 6 mm, 8 mm, and 14 mm to maintain conformability of a percentage of MPa greater than the ureter diameters based on a person's physiology.

FIG. 10 is an exemplary schematic illustration of the spiral stent showing the three diameters of 6 mm, 8 mm, and 14 mm to maintain conformability of a percentage of MPa greater than the ureter diameters based on a person's physiology.

FIG. 11 is an exemplary schematic illustration of the spiral conformal stent inserted into the kidney, ureter and bladder. This is a version that has a larger kidney spiral in the kidney to hold more firmly hold the stent in place. This adds to the conformal MPa biomorphic application of the stent to the internal area of the kidney to hold the stent in place The mechanical biology of the stent is in fact a spring that expands the ureter tube by a percentage that holds the stent in place and is conformal to the biology of the ureter tube. The ureter tube has a circumference Young's modulus of 2.31 MPa +/−0.46, whereas the stent spring has a percentage MPa greater than the ureter tube thereby holding the stent in place with a slightly larger outside spiral diameter when compared to the ureter tube. The Hook's Spring law constant of the ureter tube in that the stent is essentially a gentle spring holding the stent in place. There is a string at the end of the stent for easy removal.

FIG. 12 is an exemplary schematic illustration of the spiral conformal stent inserted into the kidney, ureter and bladder. This is a version that has a larger kidney spiral in the kidney to hold more firmly hold the stent in place. This is the “Renal Pelvis” area of the kidney that extends past the ureter into the kidney. The human Renal Pelvis is a funnel-like structure composed of a thin, distensible wall (urothelial lining supported by smooth muscle and collagenous connective tissue). Its mechanical behavior is similar to that of the proximal ureter, since the renal pelvis feeds into the ureter. As a soft biological tissue, the renal pelvis exhibits nonlinear stress-strain curves typical of collagenous tissues. This adds to the conformal MPa biomorphic application of the stent to the internal area of the kidney to hold the stent in place. The mechanical biology of the stent is in fact a spring that expands the ureter tube by a percentage that holds the stent in place and is conformal to the biology of the ureter tube. The ureter tube has a circumference Young's modulus of 2.31 MPa +/−0.46, whereas the stent spring has a percentage MPa greater than the ureter tube thereby holding the stent in place with a slightly larger outside spiral diameter when compared to the ureter tube. The Hook's Spring law constant of the ureter tube in that the stent is essentially a gentle spring holding the stent in place. There is a string at the end of the stent for easy removal.

FIG. 13 is an exemplary schematic illustration of how the spiral stent matches the peristalsis compression of the ureter while still matching the conformal MPa to hold the stent in place. Peristalsis is in the human ureter is a wave like motion that propels urine from the kidneys to the bladder the ureters which are muscular tubes contract and relax in this peristaltic motion. This process may reduce the diameter of the ureter from 10 mm to 3 mm in this process. The inventive biologic/mechanical conformability of the stent moves with this wave like motion as part of the biologic system. Additionally, since the stent is a very small diameter of 2.8 mm this compression is unaffected by the stent. Additionally, the stent has holes in it to further allow flow through the stent tube center.

FIG. 14 is an exemplary schematic illustration of the FEA (finite element analysis) 3D CAD projection of the spiral stent in the ureter tube wherein it has expanded the tube along the spiral in a conformal biology such that the 2.31 MPa of the ureter is expanded by 5% or more.

FIG. 15 is an exemplary schematic illustration of the FEA (finite element analysis) 3D CAD projection of the spiral stent along the touch points of the ureter tube wherein it has expanded the tube along the spiral in a conformal biology such that the 2.31 MPa of the ureter is expanded by 5% or more.

FIG. 16 is an exemplary schematic illustration of the FEA (finite element analysis) 3D CAD projection of the spiral stent detail calculation along the touch points of the ureter tube wherein it has expanded the tube along the spiral in a conformal biology such that the 2.31 MPa of the ureter is expanded by 5% or more.

FIG. 17 is an exemplary schematic illustration of the peristalsis contraction of the ureter and the conformability of the stent biomechanical properties.

FIG. 18 is an exemplary schematic illustration of the Fung-type model of the circumferential and longitudinal stress of the ureter tube. These calculations are the basis for the conformal biomechanical stent conformability to the 2.31 MPa Young's modulus of the ureter wall.

FIG. 19 is an exemplary schematic illustration of the Modified Mooney-Rivlin model from Rassoli et al. For circumferential stress (σ11) and longitudinal stress (σ22) of the ureter tube. These calculations are the basis for the conformal biomechanical stent conformability to the 2.31 MPa Young's modulus of the ureter wall.

FIG. 20 is an exemplary schematic detail spreadsheet of the calculations of the Modified Mooney-Rivlin model from Rassoli et al. for circumferential stress (σ11) and longitudinal stress (σ22) of the ureter tube. These calculations are the basis for the conformal biomechanical stent conformability to the 2.31 MPa Young's modulus of the ureter wall with conformal minor MPa forces of 0.40-0.89 MPa.

### DETAILED DESCRIPTION

The inventor has discovered various devices and methods for polymeric helical ureteral stents that engage with and expand the luminal wall or the ureter along most of their length to a degree such that the ureteral stent can be retained in place without the need of terminal retention elements. Such retaining engagement via controlled ureter expansion is achieved by balancing the Young's modulus and/or spring constant of the stent with the Young's modulus and/or spring constant of the ureter. In this context, it should be especially appreciated that the Young's modulus and spring constant of the stent are selected such as to allow for continuous peristalsis of the ureter, and that the risk for stone impaction between the stent and the luminal wall of the ureter is substantially eliminated. Advantageously, as the helical stent continuously abuts and extends the ureteral luminal wall and as the helical stent is formed from a tubular body, the stent will form a composite fluid path comprising an intrahelical fluid path and an intratubular fluid path.

In contrast, it should be pointed out that all or almost all of the heretofore known ureteral stents produce notable discomfort or even carry the risk of irritation or injury due relatively large retention elements in the bladder and renal pelvis, and/or lead to significant pain and ureteral spasms where a stone is located in the ureter between the stent and the luminal wall of the ureter. Compounding these undesirable features even further is the fact that the ureter exhibits continuous peristalsis, which tends to lead to unintended movement of the heretofore known stents and associated discomfort or pain, and which can exacerbate painful episodes due to impaction of stones in the ureter. Indeed, all or almost all of the heretofore know ureteral stents will not accommodate for ureteral peristalsis while maintaining placement and patency of the stent and while avoiding entrapment of calculi between the stent and luminal wall of the ureter.

In contrast, the inventor has now discovered that polymeric helical stents can be placed into and retained in the ureter of a subject in a conceptually simple and effective manner. Such placement will typically include use of a delivery device to which a ureteral stent is concentrically coupled via frictional force in a tensioned linear delivery configuration. Most typically, the ureteral stent has a tubular body having a proximal end and a distal end, and the tubular body is deformable between a tensioned linear delivery configuration and an expanded helical deployment configuration. The delivery device and stent in the tensioned linear delivery configuration are then advanced into the ureter to a delivery location (e.g., using a guide wire), and upon reaching the delivery location, the delivery device is withdrawn while the ureteral stent is maintained at the delivery location. At this point, the ureteral stent converts from the tensioned linear delivery configuration into the expanded helical deployment configuration. As will be readily appreciated, the ureteral stent in the expanded helical deployment configuration then exerts a radially expansive force on the inner wall of the ureter that radially expands the inner wall of the ureter to so retain the tubular body in the ureter while still allowing for peristalsis of the ureter as is explained in more detail below. Preferably, but not necessarily, the so deployed ureteral stent will have a relatively low number of complete helical turns, such as for example, between 4 and 10 complete helical turns over the entire length of the stent. Such low number of turns is believed to facilitate both peristaltic motion as well as motion of the ureter due to a person's movement having the stent placed in the ureter.

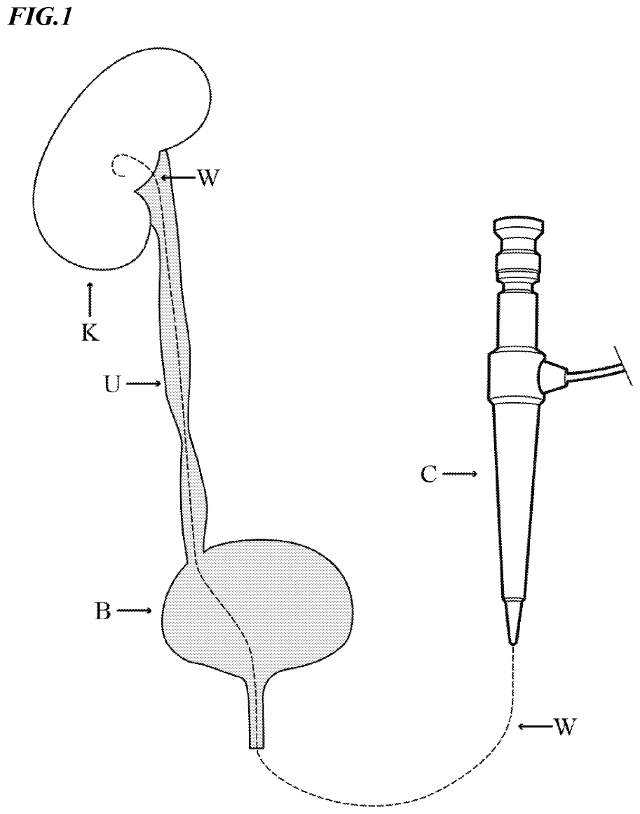

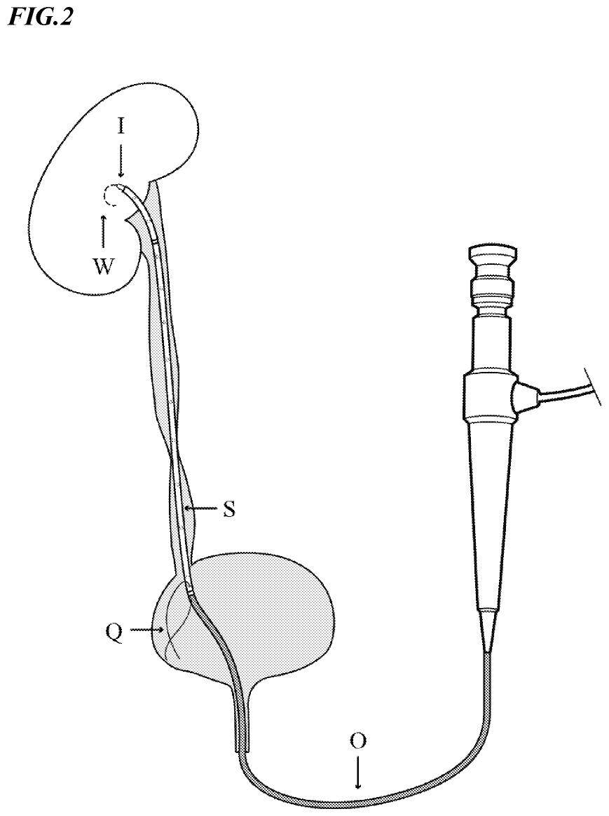

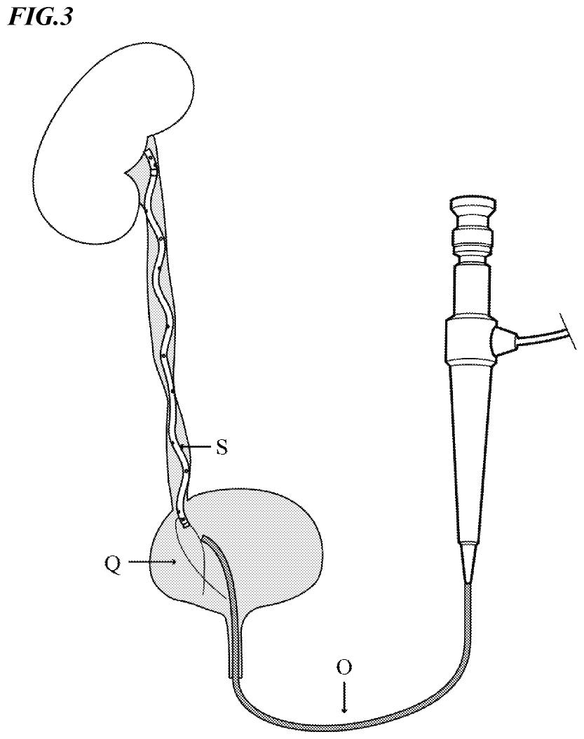

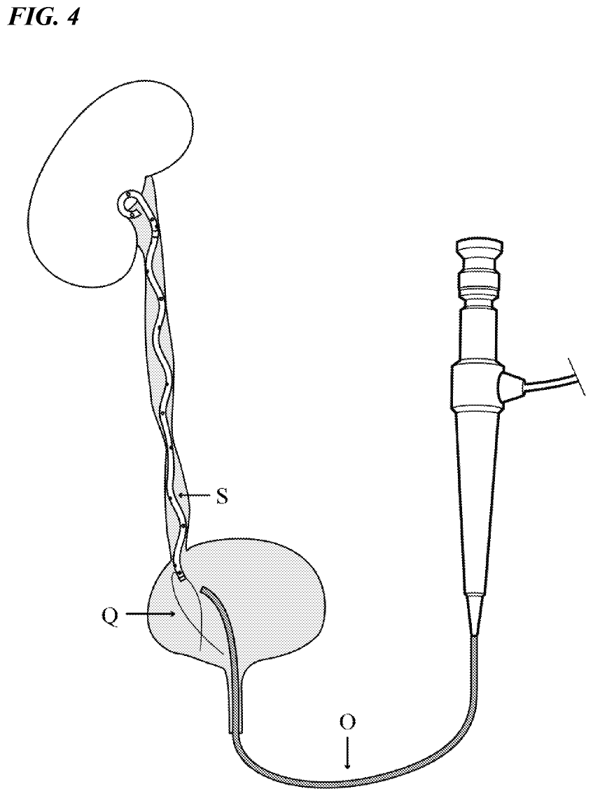

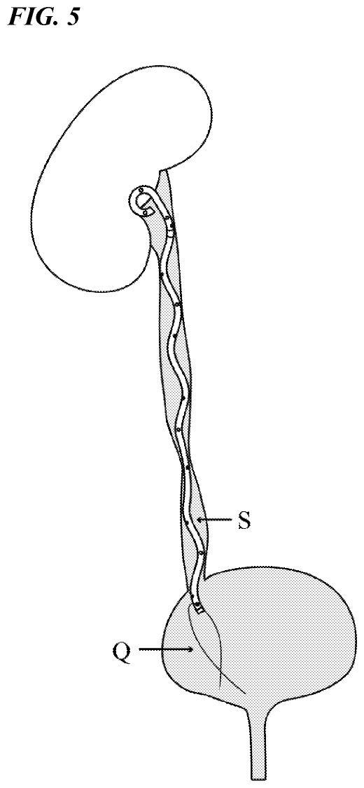

FIG. 1 exemplarily depicts a retrograde placement where guide wire W is advanced from a cystoscope C through the bladder B and ureter U/W into the renal pelvis K. Of course, it should be recognized that the guidewire may also be placed in an antegrade manner percutaneously through the kidney and renal pelvis and ureter into the bladder. Once the guidewire is in place, a delivery device is then used to advance the stent into a delivery location (typically the ureter only, possibly with the distal end extending into the bladder as is exemplarily shown in FIG. 2. Most commonly, the delivery device will comprise an inner canula over which the stent is slidingly and concentrically placed and retained by friction. Maintaining the stent in position will be achieved by use of an outer canula that during delivery abuts the distal end of the stent. Once the stent is in place, the guide wire, the inner cannula, and the outer cannula are removed. As will be readily appreciated, once the inner cannula is removed, the stent will revert from the tensioned linear delivery configuration back to the expanded helical deployment configuration as is exemplarily shown in FIG. 3. A thread Q can be coupled to the distal end of the stent for ease of later removal. FIG. 4 depicts an alternate stent that also includes a small retention element located in or near the renal pelvis. However, it should be appreciated that the retention element in most or almost all situations is not critical for retention of the stent in the ureter. FIG. 5 depicts the stent in place after removal of the delivery device and guide wire.

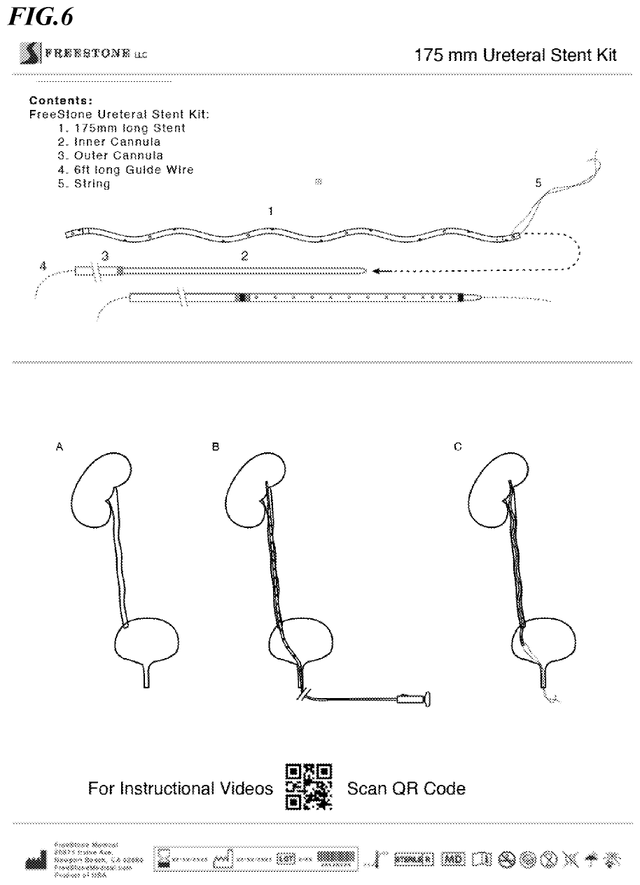

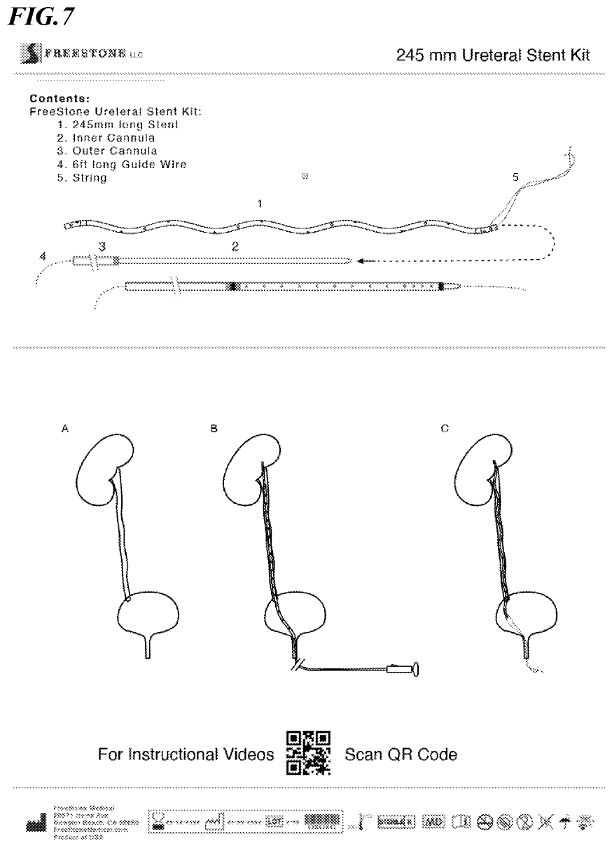

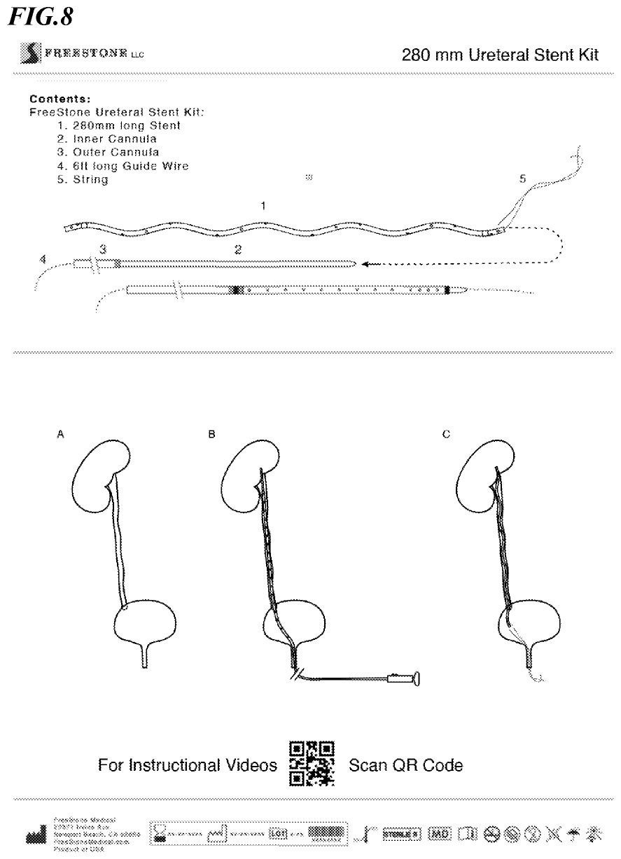

Exemplary ureteral stent kits contemplated herein are schematically shown in FIG. 6, FIG. 7, and FIG. 8. Here, three different stent lengths are shown (175 mm, 245 mm, and 280 mm, respectively), and the kit typically contains in a sterile package a stent 1 with string 5, a guide wire 4, along with an inner and outer cannula 2 and 3. The package unit will preferably also include a QR or other machine readable code that links a device to a server address that can transmit instructional content (e.g., video, tutorial, written documentation, etc.).

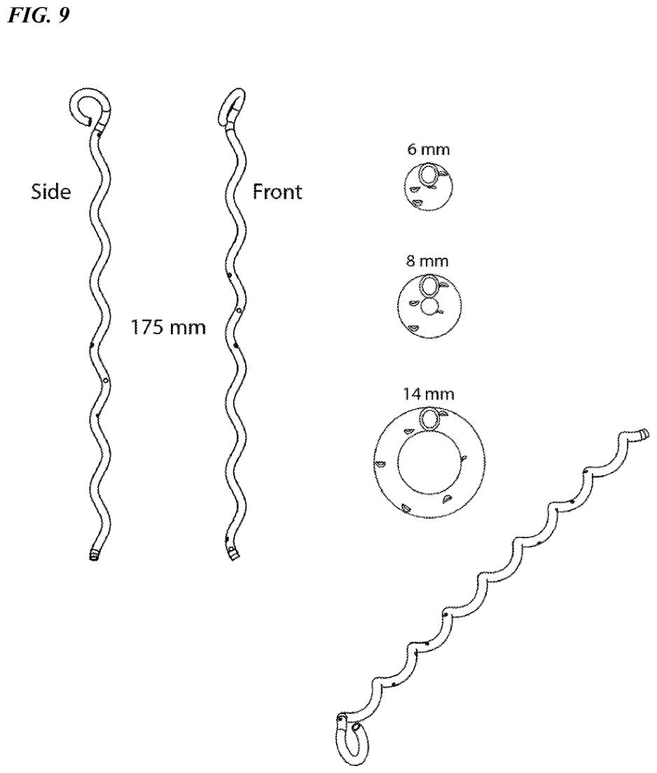

Exemplary contemplated ureteral stents are shown in more detail in FIGS. 9-11. For example, FIG. 9 depicts a relatively short (e.g., for pediatric use) ureteral stent with a small distal retention element that can be placed near or into the renal pelvis. Here, the stent is approximately 175 mm in length and has 6 complete helical turns. As can further be seen from FIG. 9, the retention element is configured as a coil where a hypothetical plane extending through the coil is at an angle (e.g., between 3 and 150 degrees, such as for example, perpendicular) relative to the longitudinal axis of the stent. As can also be readily seen, the coil need not be a full circle, but may extend to only 340 degrees, or to 320 degrees, or even less. Depending on the particular anatomical situation of the subject, contemplated stents may have varying outer helical diameters, and FIG. 9 shows three outer diameters: 6 mm, 8 mm, and 14 mm (as measured in the ‘free’ expanded helical deployment configuration without placement into the ureter and without counter pressure by the ureter). Note that the stents in the helical configuration not only provide an intratubular fluid path (space for urine to flow), but also an intrahelical fluid path. Finally, the stent of FIG. 9 has a plurality of fenestrations to facilitate flow of urine within the tubular polymeric material of the stent.

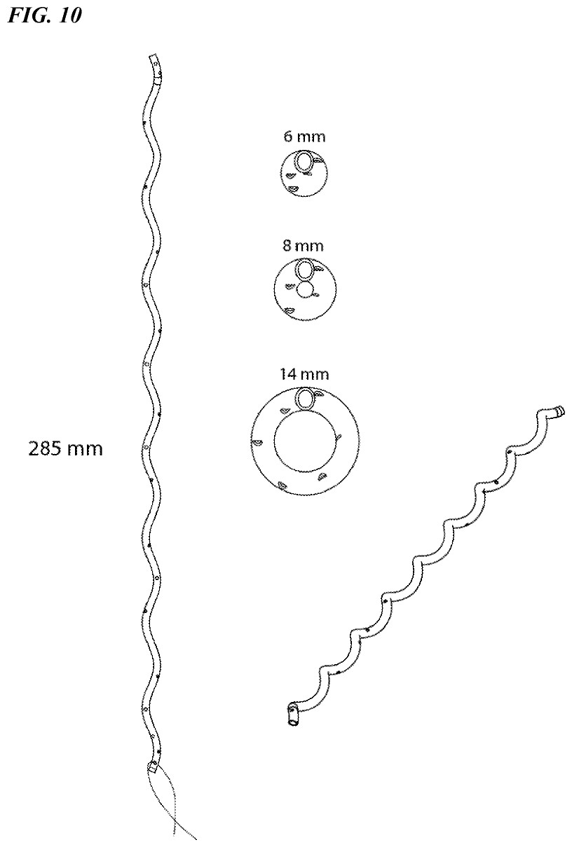

Similarly, FIG. 10 schematically illustrates a ureteral stent having a length of approximately 285 mm without any retention elements. Here the ureteral stent has 8 complete helical turns. Once more, depending on the particular anatomical situation of the subject, contemplated stents may have varying outer helical diameters, and FIG. 10 shows three outer diameters: 6 mm, 8 mm, and 14 mm (as measured in the ‘free’ expanded helical deployment configuration without placement into the ureter and without counter pressure by the ureter). As with the stent of FIG. 9 above, note that the stents in the helical configuration not only provide an intratubular fluid path (space for urine to flow), but also an intrahelical fluid path. Finally, the stent of FIG. 10 has a string for ease of retrieval.

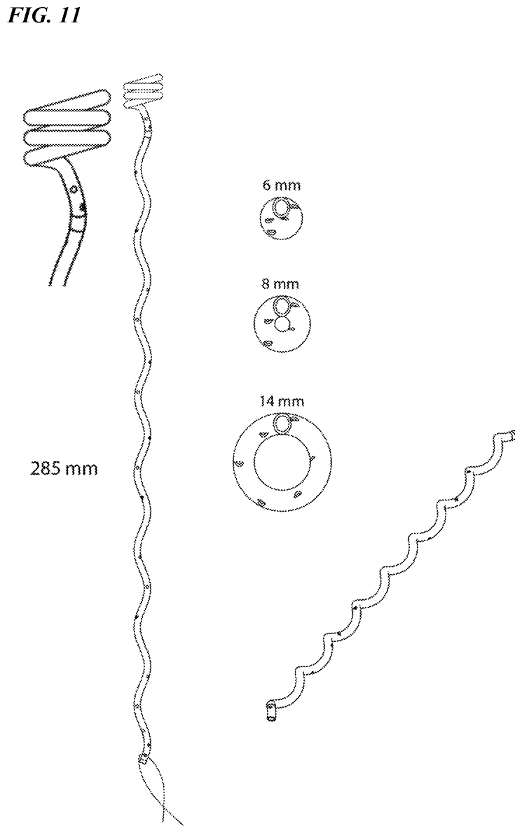

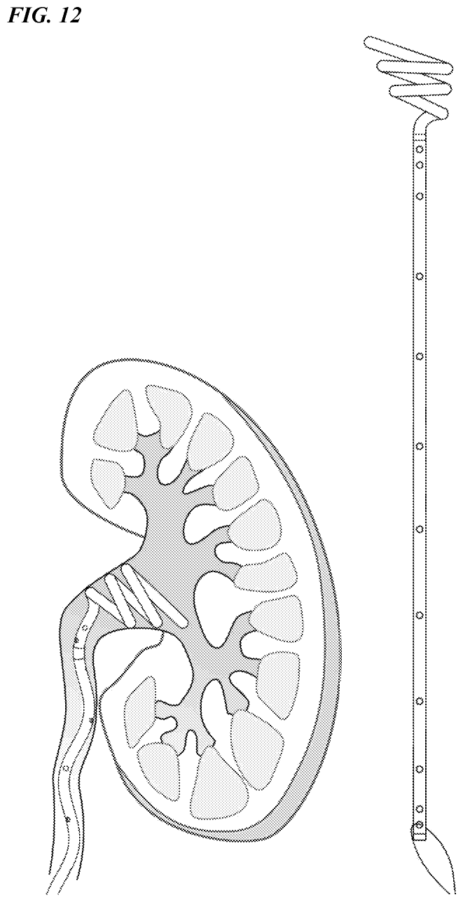

In the example of FIG. 11, the stent has a length of approximately 285 mm and has on its proximal end a plurality of coils at an angle relative to the longitudinal axis of the stent that can act as additional retention element. Here, the retention element comprises between 2 and 3 complete windings, and the exemplary stents are depicted as having three distinct outer helical diameters, 6 mm, 8 mm, and 14 mm (as measured in the ‘free’ expanded helical deployment configuration without placement into the ureter and without counter pressure by the ureter). The distal of the stent has a thread for ease of retrieval, and FIG. 12 exemplarily depicts the Stent of FIG. 11 in the ureter with the coiled retention element in the renal pelvis.

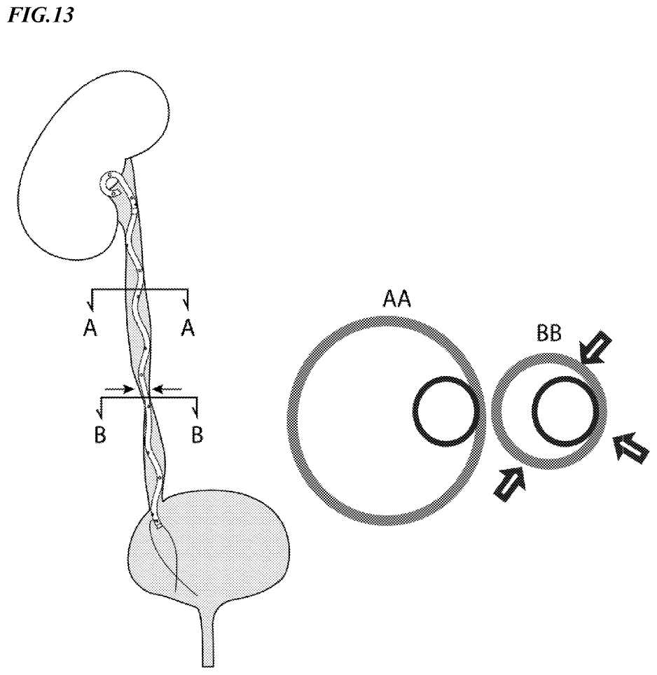

Regardless of the presence or absence of a retention element, it should be appreciated that the stent is manufactured from a material and is configured to have a Young's modulus/spring constant that is matched with the Young's modulus/spring constant of the ureter (in the radial dimension). Indeed, by the selection of the polymeric material for the stent and by the helical configuration, the stent can be manufactured such that the stent, when transitioning from the tensioned linear delivery configuration to the expanded helical deployment configuration, will exert a radial expansive (dilation) force on the ureter such that the radial expansion can be controlled. Using such control, the stent will not only be retained in its position in the ureter via radial compressive force from the ureter and radial expansive force from the stent transitioning into the expanded helical deployment configuration, but also allow for regional deformation due to peristaltic motion of the ureter. FIG. 13 schematically illustrates a helical stent according to the inventive subject matter that is placed in a ureter between the kidney and the bladder. Here, a single wave of peristaltic motion is depicted where cross section AA depicts the tubular body of the stent in a non-contracted portion of the ureter, while cross section BB depicts the tubular body of the stent in a contracted portion of the ureter. Therefore, it should be appreciated that the stents presented herein can be retained in the ureter, even without retention elements, while allowing for peristalsis. Moreover, where stones are present, the placement of the stent and intimate and continuous contact with the luminal wall of the ureter will ensure that no impaction of the stones against the luminal wall will occur, even with peristaltic movement.

In this context, it should also be noted that the stent will be configured such that the radially expansive force by the helical portion of the stent is exerted over a cumulative length of at least 70%, or at least 75%, or at least 80%, or at least 85%, or at least 90%, or at least 95% of the overall length of the stent. Thus, and viewed from a different perspective, at least 80%, or at least 85%, or at least 90%, or at least 95%, or at least 98% of the overall length of the helical portion will be in continuous contact with the luminal wall of the ureter and exert the expansive force against the luminal wall. Consequently, a predominant portion, and more typically substantially the entirety of the helical portion of the stent is retained in place and is not permitted to move, even in the presence of continuous peristaltic motion and patient movement.

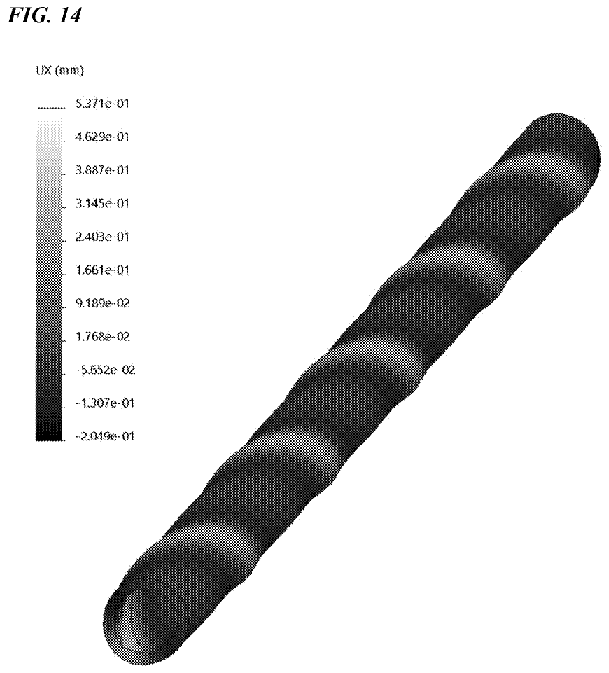

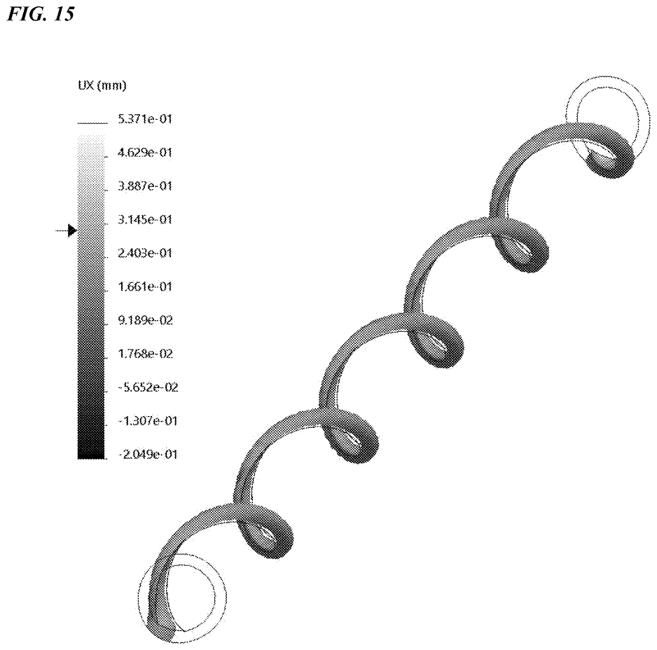

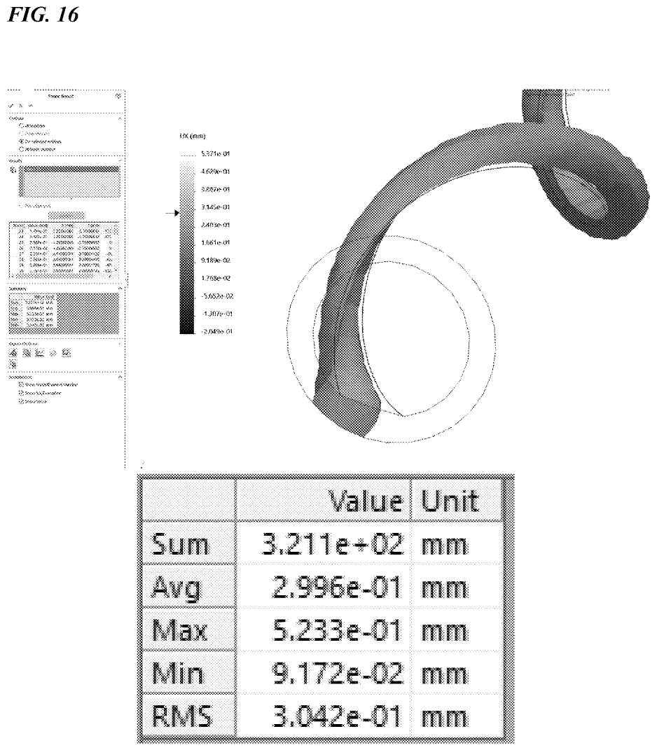

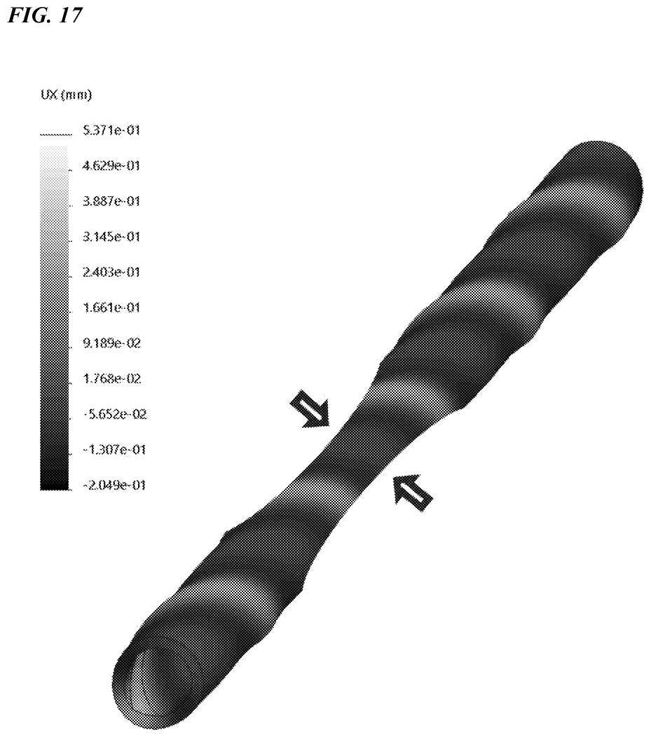

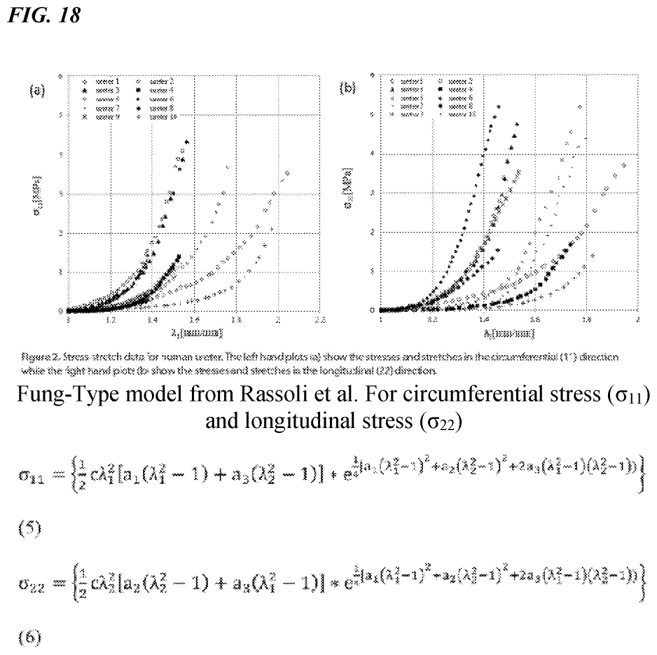

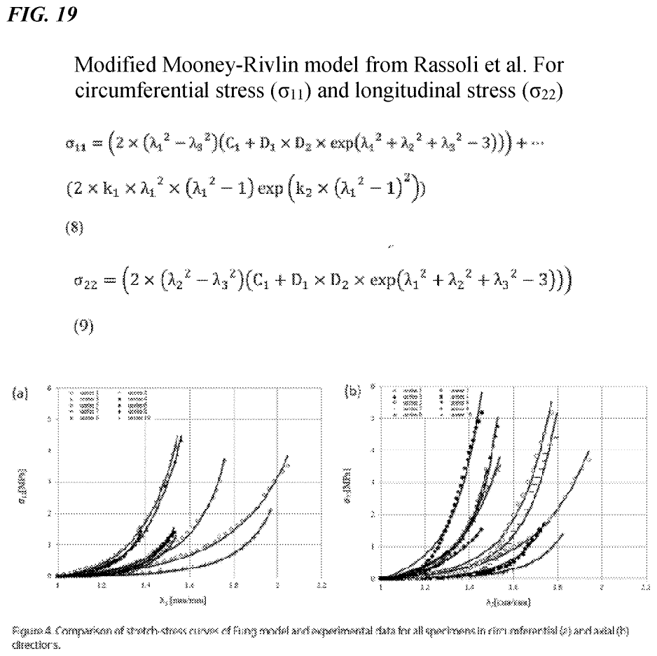

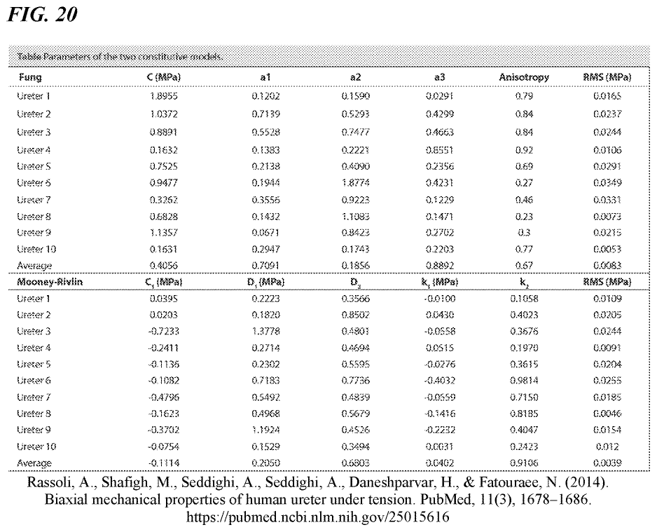

While the radially expansive forces by the stents contemplated herein may cover a relatively wide range, it is generally contemplated that the stent will be configured to radially expand the inner wall of the ureter by no more than 35%, or no more than 30%, or no more than 25%, or no more than 20%, or no more than 15%, but by at least 1%, or at least 2%, or at least 3%, or at least 4%, or at least 5%, or at least 6%, or at least 8% (as measured form the uncontracted state). For example, contemplated stents may be configured to radially expand the inner wall of the ureter by at least 2% and by no more than 8%, or by at least 4% and by no more than 10%, or by at least 6% and by no more than 12%, or by at least 4% and by no more than 12%. FIG. 14 exemplarily depicts results of a computer simulation for the elastic deformation of a ureter by a helical stent as presented herein (here shown without peristalsis) where lighter shades of gray depict expansion (up to 5 mm). FIG. 15 and the detail view of FIG. 15, shown in FIG. 16, exemplarily depict results of a computer simulation for the deformation profile of the stent of FIG. 14. Finally, FIG. 17 schematically depicts the results of a computer simulation for elastic deformation of the ureter and stent during a peristaltic contraction. As can be readily seen, the force balance between the stent and the ureter occurs substantially along the entire length of the helical portion of the stent in a purpose-driven magnitude that allows for secure retention, peristalsis, and patency of the ureter. The underlying mathematical model for the computer simulations were based on published data (Urology Journal 2014, Vol. 11, No: 3, pp1678-1686: Biaxial Mechanical Properties of Human Ureter under Tension. Rassoli et al.) and model equations were derived from the Fung type model for ureteral tissue as shown in FIG. 18 and the modified Mooney-Rivlin model for ureteral tissue in FIG. 19. Parameters for these models are provided in the Table of FIG. 20.

Therefore, it should be appreciated that the inventor has discovered that deployment of a ureteral stent can be achieved by an approach in which retention of the stent in the ureter is achieved using a predetermined/calculated balance of radial contractile force of the ureter and expansion force of compressed stent where neither side ‘wins’, but where a balance point of forces determines the amount of radial expansion. Such force balance can be expressed as (1) the ratio of the Young's modulus of the stent polymer to the Young's modulus of the luminal wall of the ureter as measured for radial expansion, and/or (2) the ratio of the spring constant of ureter in radial direction to the spring constant of the uncompressed stent in a deployed helical configuration, which will result in a limited and desired degree of radial expansion of the ureter. As will be readily appreciated, these ratios can be adjusted to achieve a desired radial expansion of the ureter.

Depending on the particular use and pathological condition, it should be appreciated that the stent can be configured to achieve a specific and desired degree of radial expansion of the ureter into which the stent is deployed. Most typically, the radial expansion will be within a range of expansion that is defined by an elastic (as opposed to plastic) deformation of the ureter. Therefore, stents can be configured to radially expand the luminal wall of the ureter by no more than 60%, or no more than 50%, or no more than 40%, or no more than 30%, or no more than 20%, or no more than 10%, or no more than 5%, or no more than 2%. Therefore, radial expansion of the luminal wall of the ureter may be between 1-5%, or between 2-8%, or between 5-12%, or between 10-20%, or between 15-30%, or between 20-40%. In that context, it should be noted that radial expansion is relative to an age-adjusted physiological normal (healthy) ureteral diameter. For example, an age-adjusted physiological normal non-contracted ureter diameter for a 30 year-old male is between 6 mm and 10 mm. As will be readily appreciated, the ureteral diameter of a patient can be measured, and the proper stent parameters can then be selected accordingly.

Based on published experimental data, the Young's modulus in radial direction for a human ureter can generally be assumed to be between 2.0 and 2.5 mPa. Therefore, to accommodate various pathological conditions and inter-patient variability, the inventor contemplates that the Young's modulus of the polymeric material can be between 1.0 mPa and 2.0 mPa, or between 1.5 mPa and 3.0 mPa, or between 2.5 mPa and 4.5 mPa, or between 3.5 mPa and 6.5 mPa, and in some cases even higher. Consequently, suitable ratios of the Young's modulus of the polymeric material to the Young's modulus of the ureter for radial expansion may be between 1.1:1 and 1.5:1, or between 1.2:1 and 1.9:1, or between 1.4:1 and 2.2:1, or between 1.6:1 and 2.5:1. Therefore, and viewed from a different perspective, the ratio of the Young's modulus of the polymeric material to the Young's modulus of the ureter for radial expansion may be selected such that, upon deployment of the stent, an inner wall diameter of the ureter is increased by between 2% and 5%, or between 3% and 7%, or between 3% and 10%, or between 5% and 12%, or even higher.

Similarly, when considering the respective spring constants, it is contemplated that the ratio of the spring constant for radial expansion of the ureteral stent and the spring constant for radial expansion of the ureter may be between 2:1 and 5:1, or between 2.5:1 and 7:1, or between 3:1 and 10:1, or between 4:1 and 15:1. For example, contemplated spring constants for radial expansion of the ureter may be between 1,000 N/m and 10,000 N/m, or between 2,000 N/m and 14,000 N/m, or between 5,000 N/m and 20,000 N/m, or between 8,000 N/m and 25,000 N/m, and/or contemplated spring constants for the radial expansion of the ureteral stent may be between 12,000 N/m and 20,000 N/m, or between 18,000 N/m and 30,000 N/m, or between 25,000 N/m and 40,000 N/m, or between 30,000 N/m and 60,000 N/m.

Therefore, and viewed from a different perspective, the Young's modulus of the ureter and the Young's modulus of the material for the stent, and/or the spring constant of the ureter and the spring constant of the stent can be adjusted relative to each other to achieve a specific radial expansion. For example, the ratio of the Young's modulus of the polymeric material to the Young's modulus of the ureter for radial expansion may be selected such that, upon deployment of the stent, the inner wall diameter of the ureter is increased by between 2% and 8%, or by between 3% and 10%, or by between 5% and 15%, or by between 10% and 20%, or by between 15% and 35%, or even more. Therefore, a suitable Young's modulus for the polymeric material of the helical stents contemplated herein is between 10.0 MPa and 6.8 MPa, or between 8.0 MPa and 5.0 MPa, or between 4.0 MPa and 1.2 MPa, or between 3.0 MPa and 0.8 MPa, or between 2.0 MPa and 0.3 MPa. Viewed from a different perspective, the ratio of the Young's modulus of the polymeric material of the helical portion to the Young's modulus of the ureter for radial expansion may be between 1.1:1 and 1.6:1, or between 1.2:1 and 1.9:1, or between 1.8:1 and 2.5:1, and even higher.

Alternatively, of additionally, it is contemplated that the ratio of a spring constant for radial expansion of the ureteral stent and the spring constant for radial expansion of the ureter is between 1.5:1 and 3.5:1, or between 2:1 and 5:1, or between 3:1 and 10:1, or between 7:1 and 20:1, or even higher. Most typically, however, the spring constant for radial expansion of the ureter will be between 300 N/m and 3,000 N/m, or between 1,000 N/m and 10,000 N/m, or between 7,000 N/m and 25,000 N/m, and/or the spring constant for radial expansion of the ureteral stent will be between 5,000 N/m and 15,000 N/m, or between 10,000 N/m and 30,000 N/m, or between 30,000 N/m and 60,000 N/m.

While the above considerations and numerical values are generally contemplated for many embodiments, it should be recognized that alternative configurations are also deemed suitable for use herein and include those with mechanically stiffer materials (higher Young's modulus) that have a lower number of coils, and softer materials (lower Young's modulus) with a higher number of coils. As such, stents having equivalent ratios of Young's modulus to number of coils are also expressly contemplated.

As will be readily appreciated, the helical stents contemplated herein may have a variety of dimensions, which will typically depend on the patient's age and gender as well as the condition to be treated. Most typically, however, suitable stents will have an outer diameter of between 2 mm (6 French) and 5 mm (15 French) in the tensioned linear delivery configuration, such as an outer diameter of 2.67 mm (8 French), or 4.0 mm (12 French), or 4.67 mm (14 French) in the tensioned linear delivery configuration. Most typically, but not necessarily, contemplated stents may have a length between 150 mm and 300 mm in the linear delivery configuration (e.g., at least 160 mm).

With further regard to the helical deployment configuration, it is generally contemplated that the stent forms a helix with an inner open diameter of between 1 mm and 8 mm (e.g., 2 mm, 3 mm, 4 mm, 5 mm, 6 mm, 7 mm), and/or an outer helical diameter of between 5 mm and 16 mm such as between 5-7 mm, or 7-9 mm, or 9-11 mm, or 11-13 mm). In still further contemplated aspects, the stents presented herein will have a relatively low number of complete helical turns, such as for example between 3 and 15 or more typically between 4 and 12 complete helical turns in the helical deployment configuration. In this context, it should be appreciated that the helical turns will sufficiently spaced apart such that when the inner diameter of the ureter changes (e.g., from 8 mm to 2 mm) during passing of a peristaltic wave, the stent will allow this change in inner diameter without inhibiting peristalsis. For example, spacing between complete turns (in the helical deployment configuration) may be between 20 mm to 28 mm, or between 27 mm to 30 mm, or between 30 mm to 35 mm, or between 32 mm to 37 mm, or between 35 mm to 40 mm, or between 37 mm to 42 mm, or between 45-50 mm, or between 37 mm to 55 mm, and even more. Therefore, exemplary ureteral stents may have between 4 and 7 complete helical turns and a length of between 150-200 mm, or between 5 and 8 complete helical turns and a length of between 220-270 mm, or between 6 and 9 complete helical turns and a length of between 250-300 mm. Moreover, it should also be appreciated that the stent, in the helical deployment configuration, will form a composite fluid path comprising an intrahelical fluid path and an intratubular fluid path.

As can also be seen from some of the figures, contemplated stents may have a plurality of fenestrations that may assist urine flow in the intratubular fluid path. Most typically, the fenestrations will be circular openings, but may also have other geometries. In still further contemplated aspects, it should be noted that the fenestrations need not be homogenously distributed along the longitudinal direction of the stent, but that the distal portion of the stent may have a larger number of fenestrations as compared to the proximal portion to so allow for an increased drainage capacity for the portion of the stent that extends towards and/or into the bladder. For example, the number of fenestrations may be at least 5%, or at least 10%, or at least 15%, or at least 20%, or at least 33%, or at least 50%, or at least 75%, or at least 100% higher in the distal portion as compared to the proximal portion. In this context, it should be noted that the distal portion of the stent is the portion that extends from the distal end of the stent towards the middle of the stent over no more than 50%, or no more than 40%, or no more than 30%, or no more than 20% of the total length of the stent.

In this context, it should be appreciated that the helical ureteral stent comprises a material (typically a polymeric material) and is formed such that the helical stent is elastically deformable from a helical configuration to a linear configuration to so allow for delivery on a stent delivery device. In most typical embodiments, the stent delivery device will include a channel that can accommodate a guide wire such that the stent delivery device and the stent can be advanced over the wire to the desired location in the ureter. To that end, the stent delivery device may also include a radiopaque marker to assist proper positioning in the ureter. As should be further appreciated, the stent may or may not include one or more radiopaque markers on the outside surface of the stent, and where present, the stent will typically have a radiopaque marker on or proximal to the end sections. Alternatively, the polymeric material for the stent may also comprise a radiopaque salt such as BaSO₄, Bi₂O₃, or metal such as tungsten or tantalum to aid in visualization.

In particular, the helical stent is threaded over a portion of the inner cannula that extends beyond the outer cannula, thereby elastically deforming the helical configuration into a tensioned linear delivery configuration. In this context it should be especially noted that the Young's modulus of the material for the helical stent is not only effective to exert an expansive radial force onto the luminal wall of the ureter, but also effective to exert a frictional force on the outside surface of the inner cannula of the delivery device in the tensioned linear delivery configuration to so retain the stent on the delivery device. Such frictional force is especially advantageous as this will allow the physician to manipulate the stent delivery device and stent in the various environments encountered during retrograde or percutaneous antegrade delivery without loss or premature deployment of the stent. Once the stent is located in the proper or desired location, as for example verified by fluoroscopy, the guide wire can be withdrawn, and the inner cannula retracted through the outer cannula to thereby disengage from the inner cannula and to elastically return to a helical deployment configuration.

As will be readily appreciated, contemplated stents will preferably be manufactured from a polymeric material or composite polymeric material (mixtures or heteropolymers), and especially suitable polymeric materials include low density polyethylene, high density polyethylene, polyethylene terephthalate, polyurethane, nylon, polyamide, polycarbonate, poly(ethylene-co-vinyl acetate) (PEVA), poly(n-butyl methacrylate) (PBMA), and all reasonable mixtures thereof. In addition, it is also contemplated that suitable stents may be manufactured from a biodegradable or bio-erodible/absorbable material such as polylactic acid, polyhydroxy butyric acid, tyrosine-derived polycarbonate polymers, etc.

As already noted above, contemplated stents may further include one or more retention elements at respective ends. The retention elements may be configured in a variety of manners, however, it is typically preferred that the retention element has a ‘pigtail’ configuration, a partial or complete coil, a stacked coil, or a loop configuration. Moreover, where a coil-type retention element is used, it is generally preferred that the coil will have a hypothetical plane that is at an angle to a hypothetical longitudinal axis of the helical stent. Suitable coils for the stents contemplated herein may have a constant diameter, and more preferably an increasing diameter as is exemplarily shown in the stent of FIG. 12.

Most typically, contemplated stents will be used for temporary stenting, and typical dwell times for a stent will therefore be between 1-3 days, or between 3-7 days, between 1-2 weeks, or between 2 and 4 weeks, and in some cases even longer, and particularly (but not necessarily) where the stent comprises a pharmaceutical agent that may be fixed or elutable. For example, contemplated pharmaceutical agents include one or more antimicrobial agents (such as antifungal agents, antibiotic agents, bacteriostatic agents, etc.), one or more chemotherapeutic agents (antineoplastic agents, cytotoxic agents, cytostatic agents, etc.), one or more anti-inflammatory agents (steroids, NSAIDS, etc.), immune modulatory agent (cytokines, chemokines, etc.), and/or one or more radioactive agent (preferably alpha emitters). Most typically, the pharmaceutical agent will be coated or otherwise coupled to the stent in a manner that allows controlled delivery from the stent to the ureter and downstream tissues. For example, the pharmaceutical agent may be disposed in a dissolving, biodegradable, or erodible matrix that is coated onto the stent and that releases the agent over time. Alternatively, the stent may comprise a porous material or have at least some fenestrations or other void spaces that are filled with the pharmaceutical agent (which may be disposed in a controlled release composition) for timed release. As will be readily recognized, the type of pharmaceutical agent and medical condition will at least in part determine the release characteristics, and the skilled artisan will be apprised of such parameters and be able to select the proper materials for controlled release of the agent.

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . . and N, the text should be interpreted as requiring only one element from the group, not A plus N, or B plus N, etc.

What is claimed is:

1. A ureteral stent, comprising:
a tubular body having a proximal portion with a proximal end and a distal portion with a distal end;
wherein the tubular body is deformable between a tensioned linear delivery configuration and an expanded helical deployment configuration;
wherein the tubular body comprises a polymeric material having a Young's modulus and/or wherein a helical portion has a spring constant that is matched to a spring constant for radial expansion of a ureter effective to
(a) exert a frictional force on a delivery device in the tensioned linear delivery configuration to so retain the tubular body on the delivery device; and
(b) exert a radially expansive force on an inner wall of the ureter in the expanded helical deployment configuration to radially expand the inner wall of the ureter to so retain the tubular body in the ureter; and
optionally a plurality of fenestrations in the tubular body.

2. The ureteral stent of claim 1, wherein the proximal and/or distal portion further comprises a non-helical portion, optionally wherein the non-helical portion comprises a retention element configured for placement into a pelvis of a kidney or a bladder, and/or optionally wherein the non-helical portion comprises, upon placement in the pelvis of the kidney, a coil configuration that is perpendicularly oriented to a longitudinal axis of the helical portion.

3. The ureteral stent of claim 1, wherein the stent exerts the radially expansive force over a cumulative length of at least 75% of an overall length of the stent.

4. The ureteral stent of claim 1, wherein the stent is configured to radially expand the inner wall of the ureter by at least 2% and by no more than 8%.

5. The ureteral stent of claim 1, wherein a ratio of the Young's modulus of the polymeric material to a Young's modulus of the ureter for radial expansion is between 1.2:1 and 1.9:1.

6. The ureteral stent of claim 1, wherein a ratio of the Young's modulus of the polymeric material to a Young's modulus of the ureter for radial expansion is selected such that, upon deployment of the stent, an inner wall diameter of the ureter is increased by between 3% and 10%.

7. The ureteral stent of claim 1, wherein the Young's modulus of the polymeric material is between 2.5 MPa and 4.5 MPa.

8. The ureteral stent of claim 1, wherein a ratio of the spring constant for radial expansion of the ureteral stent and a spring constant for radial expansion of the ureter is between 3:1 and 10:1.

9. The ureteral stent of claim 1, wherein a spring constant for radial expansion of the ureter is between 2,000 N/m and 10,000 N/m, and/or wherein the spring constant for radial expansion of the ureteral stent is between 15,000 N/m and 30,000 N/m.

10. The ureteral stent of claim 1, wherein the stent does not comprise a retention element extending away from the tubular body.

11. The ureteral stent of claim 1, wherein the stent has a length of at least 160 mm in the linear delivery configuration.

12. The ureteral stent of claim 1, wherein the stent has between 4 and 10 complete helical turns in the helical deployment configuration.

13. The ureteral stent of claim 1, wherein the stent has between 4 and 6 complete helical turns and a length of between 150-200 mm, or wherein the stent has between 5 and 7 complete helical turns and a length of between 220-270 mm, or wherein the stent has between 6 and 8 complete helical turns and a length of between 250-300 mm in the helical deployment configuration.

14. The ureteral stent of claim 1, wherein the stent, in the helical deployment configuration, forms a composite fluid path comprising an intrahelical fluid path and an intratubular fluid path.

15. The ureteral stent of claim 1, wherein the polymeric material further comprises a pharmaceutical agent, optionally selected from the group consisting of an antimicrobial agent, a chemotherapeutic agent, an anti-inflammatory agent, an immune modulatory agent, and a radioactive agent.

16. A method of placing a ureteral stent, comprising:
providing a delivery device to which a ureteral stent is concentrically coupled via frictional force in a tensioned linear delivery configuration;
wherein the ureteral stent has a tubular body having a proximal end and a distal end, and wherein the tubular body is deformable between the tensioned linear delivery configuration and an expanded helical deployment configuration;
advancing the delivery device and stent in the tensioned linear delivery configuration into the ureter to a delivery location;
upon reaching the delivery location, withdrawing the delivery device while maintaining the ureteral stent at the delivery location to thereby convert the ureteral stent from the tensioned linear delivery configuration into the expanded helical deployment configuration;
wherein the ureteral stent in the expanded helical deployment configuration exerts a radially expansive force on an inner wall of a ureter that radially expands the inner wall of the ureter to so retain the tubular body in the ureter while allowing for peristalsis of the ureter.

17. The method of claim 16, wherein the delivery device is fed over a guide wire.

18. The method of claim 16, wherein the ureteral stent is a ureteral stent according to claim 1.

19. The method of claim 16, wherein the step of advancing comprises retrograde advancing of the delivery device and stent through the urethra and bladder.

20. The method of claim 16, wherein the step of advancing comprises percutaneous antegrade advancing of the delivery device and stent through the kidney.
